(19) United States
(12) Patent Application Publication
(10) Pub. No.: US 2005/0158369 A1
(43) Pub. Date: Jul. 21, 2005

(54) Starch-containing cosmetic wipes

(75) Inventors: Albrecht Dorschner, Hamburg (DE); Jorg Kuther, Halstenbek (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/962,974

(22) Filed: Oct. 12, 2004

Related U.S. Application Data
(63) Continuation of application No. PCT/EP03/03610, filed on Apr. 8, 2003.

(30) Foreign Application Priority Data
Apr. 11, 2002 (DE) 102 16 511.4

(51) Int. Cl. A61K 7/06

(52) U.S. Cl. 424/443; 424/70.13

(57) ABSTRACT

The present invention is a cosmetic preparation-containing composition and methods of making thereof. The composition comprises a water-insoluble substrate impregnated with a cosmetic preparation comprising one or more pregelatinized, crosslinked starch derivatives. In one embodiment, the composition is a wipe that can appear either wet or dry.

### CROSS-REFERENCE TO RELATED APPLICATIONS

This is a continuation application of PCT/EP03/03610, filed Apr. 8, 2003, which is incorporated herein by reference in its entirety, and also claims the benefit of German Priority Application No. 102 16 511.4, filed Apr. 11, 2002.

### FIELD OF THE INVENTION

The present invention relates to a water-insoluble substrate impregnated with a cosmetic preparation comprising one or more pregelatinized, crosslinked starch derivatives.

### BACKGROUND OF THE INVENTION

The desire to look beautiful and attractive is naturally rooted in humans. Although the beauty ideal has undergone changes over the course of time, the achievement of a flawless outer appearance has always been the aim of humans. An essential part of a beautiful and attractive appearance is the condition and appearance of the skin.

The skin is the largest human organ. Amongst its many functions (for example temperature regulation and as a sensory organ), the barrier function, which prevents the skin (and thus ultimately the entire organism) from drying out, is probably the most important. At the same time, the skin acts as a protective device against the penetration and absorption of external substances. This barrier function is effected by the epidermis which, being the outermost layer, forms the actual protective sheath against the environment. Being about one tenth of the total thickness, it is also the thinnest layer of the skin.

In order that the skin can completely perform its biological functions, it requires regular cleansing and care and also protection against UV radiation. Cleansing of the skin serves here to remove dirt, perspiration and remains of dead skin particles, which form an ideal breeding ground for pathogens and parasites of all types. Skincare products, generally creams, ointments or lotions, serve mostly to moisturize and re-fat the skin. Active ingredients are often added to them which are intended to regenerate the skin and, for example, prevent and reduce its premature aging (e.g. the formation of wrinkles, lines). To protect against harmful UV radiation from sunlight, UV light protection filters are added to many cosmetic and dermatological skincare products. Specific sunscreen products, whose primary task is protection against UV radiation, are also supplied in a large product range.

Most cosmetic and dermatological preparations are in the form of more or less viscous liquids. These high-viscosity preparations (ointments, creams, gels) or low-viscosity preparations (lotions, washes) are generally removed from their packagings and applied to the skin using the hands or fingers.

A particular application form of cosmetic and/or dermatological preparations is wipes. These may already be impregnated with the cosmetic and/or dermatological agent by the manufacturer and thus have the advantage that the preparation in them is already in the correct dose. Furthermore, they avoid the disadvantage of preparations stored in bottles and pots, the packaging of which can be broken and the contents of which can “run out”. A further disadvantage of bottles and pots compared with wipes is the fact that, as they are removed, their contents are contaminated with microorganisms as a result of contact with hand and fingers.

However, a disadvantage of the prior art is the fact that it has hitherto not been possible to produce wipes with a cosmetic preparation comprising starch or derivatives thereof on an industrial scale. Starch and its derivatives produce a pleasantly smooth, silky feel on the skin during and following application of corresponding cleansing and care products and is therefore an often used constituent of cosmetic preparations. According to the prior art, starch or its derivatives are virtually insoluble in cosmetic preparations and, at best, are soluble in small amounts. Numerous experiments to spray wipes with a starch (derivative)-containing preparation led, as a result of these compounds precipitating out in the spraying plants, to blockages of the spray nozzles and, thus, to unevenly impregnated wipes and/or functionally inefficient impregnation and spraying plants.

Another method of producing impregnated wipes consists in immersing the wipe into the cosmetic preparation. However, conventional starch (derivative)-containing preparations have too high a viscosity for wipes to be impregnated by immersion into these preparations, thereby uniformly impregnating with product-relevant amounts of the solutions.

### SUMMARY OF THE INVENTION

It was therefore the object of the present invention to overcome the shortcomings of the prior art and to develop starch (derivative)-containing cosmetic wipes which can be produced using the conventional methods for producing such wipes on an industrial scale.

Surprisingly, the object is achieved by a water-insoluble substrate impregnated with a cosmetic preparation comprising one or more pregelatinized, crosslinked starch derivatives.

The products according to the invention are characterized by an extraordinarily pleasant smooth and silky feel on the skin during and after application. Due to their physicochemical properties (solubility, rheology), the cosmetic preparations according to the invention can, surprisingly, either be sprayed onto the substrates according to the invention, or be wetted in an immersion bath without problems.

Although U.S. Pat. No. 6,248,338 describes aqueous surfactant systems with pregelatinized, crosslinked starch derivatives, this specification was unable to point the way to the present invention.

According to the invention, preference is given to the use of wipes as water-insoluble substrate. The wipes advantageous according to the invention can be smooth or else surface-structured. It is possible to use closed or perforated wipes, and also gauzes. According to the invention, preference is given to surface-structured wipes. Besides wipes, pads and balls can also be used advantageously according to the invention.

### DETAILED DESCRIPTION OF THE PREFERRED EMBODIMENTS

According to the invention, “dry” substrates are preferably used in combination with the preparations according to the invention, said substrates consisting of nonwoven material, in particular of water-jet-consolidated and/or water-jet-impressed nonwoven.

Such nonwovens can have macro impressions in any desired pattern. The choice to be made depends firstly on the impregnation to be applied and secondly on the field of use in which the subsequent substrate is to be used.

If impressed or perforated nonwovens are used, then large cavities on the surface of the nonwoven and within the nonwoven facilitate the uptake of dirt and contaminations when the impregnated substrate is passed over the skin. The cleansing effect can be increased many times over relative to the unimpressed substrate.

It has proven advantageous for the substrate if it has a weight of from 30 to 120 g/m², preferably from 40 to 80 g/m² and particularly preferably 45 to 60 g/m² (measured at 20° C.±2° C. and at a room air humidity of 65%±5% for 24 hours).

The thickness of a nonwoven according to the invention is 0.3 to 5 mm, preferably 0.4 mm to 2 mm, in particular 0.6 mm to 0.9 mm.

Starting materials for the water-insoluble substrate which can be used are generally all organic and inorganic natural and synthetic based fiber materials. Examples which may be given are viscose, cotton, cellulose, jute, hemp, sisal, silk, wool, polypropylene, polyester, polyethylene terephthalate (PET), aramid, nylon, polyvinyl derivatives, polyurethanes, polylactide, polyhydroxyalkanoate, cellulose esters and/or polyethylene and also mineral fibers, such as glass fibers or carbon fibers. However, the present invention is not limited to the materials specified, it being possible to use a large number of further fibers for forming the nonwoven. For the purposes of the present invention, it is particularly advantageous if the fibers used are not water-soluble.

In a particularly advantageous embodiment of a nonwoven, the fibers consist of a mixture of 60 to 80% viscose with 40 to 20% PET in particular 70% viscose and 30% PET.

Also particularly advantageous are fibers of high-strength polymers, such as polyamide, polyesters, and/or highly drawn polyethylene.

Moreover, the fibers can also be colored in order to be able to emphasize and/or enhance the visual attractiveness of the nonwoven. The fibers can additionally comprise UV stabilizers and/or preservatives.

The fibers used to form the substrate preferably have a water-absorption rate of more than 60 mm/[10 min] (measured using the EDANA test 10.1-72), in particular more than 80 mm/[10 min].

Furthermore, the fibers used to form the substrate preferably have a water-absorption rate of more than 5 g/g (measured using the EDANA test 10.1-72), in particular more than 8 g/g.

Advantageous substrates for the purposes of the present invention have a tear strength of, in particular,

| | | [N/50 mm] |
|---|---|---|
| in the dry state | machine direction | >60, preferably >80 |
| | transverse direction | >20, preferably >30 |
| in the impregnated | machine direction | >4, preferably >60 |
| state | | |
| | transverse direction | >10, preferably >20 |

The expandability of advantageous substrates is preferably

| | | |
|---|---|---|
| in the dry state | machine direction | 15% to 100%, preferably |
| | | 20% and 50% |
| | transverse direction | 40% to 120%, preferably |
| | | 50% and 85% |
| in the impregnated | machine direction | 15% to 100%, preferably |
| state | | 20% and 40% |
| | transverse direction | 40% to 120%, preferably |
| | | 50% and 85% |

According to the invention, substrates impregnated with a cosmetic preparation advantageously appear either wet or else appear dry.

For the substrates according to the invention which are not dried after impregnation, it is advantageous according to the invention if the weight ratio of wipe to cosmetic preparation is 0.7:1 to 1:4. Even larger amounts of cosmetic preparation are advantageous according to the invention. The degree of impregnation of the substrate is thus advantageously ≧100% by weight.

For substrates according to the invention which are dried after impregnation, a weight ratio of from 1:1 to 1:4 after impregnation and before drying is particularly advantageous according to the invention. In the case of these substrates too, larger amounts of cosmetic preparation are advantageous according to the invention. The amount of vaporizable constituents in the cosmetic preparation is more likely kept low due to the subsequent drying, i.e. preparations with a relatively high active ingredient concentration are used. The degree of impregnation of the substrate is advantageously ≧150% by weight and less than/equal to 300% by weight.

For the purposes of the present invention, it is advantageous if the cosmetic preparation according to the invention with which the water-insoluble substrate is impregnated comprises one or more pregelatinized, crosslinked starch derivatives in a concentration of from 0.1 to 20% by weight, particularly preferably in a concentration of from 0.3 to 15% by weight and very particularly preferably in a concentration of from 0.5 to 10% by weight, based on the total weight of the cosmetic preparation in the composition, with which this is applied to the substrate during impregnation.

It is advantageous according to the invention if the pregelatinized, crosslinked starch derivatives used are hydroxypropylated phosphate esters. Those starch derivatives as described in U.S. Pat. No. 6,248,338, particularly advantageously hydroxypropyl distarch phosphate (CAS number 113894-92-1), are particularly advantageous. Very particular preference is given here to the use of an hydroxypropyl distarch phosphate as is sold as the product Structure® XL from National Starch.

According to the invention, the cosmetic preparation according to the invention can advantageously comprise further cosmetic and/or dermatological active ingredients, auxiliaries and/or additives. The quantitative data given in this description refer to the concentrations as are present in the preparation before it is applied to the wipe.

Advantageous washing-active anionic surfactants for the purposes of the present invention are, for example,

acylamino acids and salts thereof, such as

acyl glutamates, in particular sodium acyl glutamate
sarcosinates, for example myristoyl sarcosine, TEA lauroyl sarcosinate, sodium lauroyl sarcosinate and sodium cocoyl sarcosinate,
   sulfonic acids and salts thereof, such as
acyl isethionates, e.g. sodium/ammonium cocoyl isethionate,
sulfosuccinates, for example dioctyl sodium sulfosuccinate, disodium laureth sulfosuccinate, disodium lauryl sulfosuccinate and disodium undecyleneamido MEA sulfosuccinate,
   and sulfuric esters, such as
alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Advantageous washing-active cationic surfactants for the purposes of the present invention are quaternary surfactants. Quaternary surfactants contain at least one N atom which is covalently bonded to 4 alkyl or aryl groups. Benzalkonium chloride, alkylbetaine, alkylamidopropylbetaine and alkylamidopropylhydroxysultaine are advantageous.

Advantageous washing-active amphoteric surfactants for the purposes of the present invention are
acyl/dialkylethylenediamines, for example sodium acyl amphoacetate, disodium acyl amphodipropionate, disodium alkyl amphodiacetate, sodium acyl amphohydroxypropylsulfonate, disodium acylamphodiacetate and sodium acyl amphopropionate.

Advantageous washing-active nonionic surfactants for the purposes of the present invention are
alkanolamides, such as cocamides MEA/DEA/MIPA,
esters which are produced by the esterification of carboxylic acids with ethylene oxide, glycerol, sorbitan or other alcohols,
ethers, for example ethoxylated alcohols, ethoxylated lanolin, ethoxylated polysiloxanes, propoxylate POE ethers and alkyl polyglycosides, such as lauryl glucoside, decyl glycoside and cocoglycoside.
   Further advantageous anionic surfactants are
taurates, for example sodium lauroyl taurate and sodium methyl cocoyl tau rate,
ether carboxylic acids, for example sodium laureth-13 carboxylate and sodium PEG-6 cocamide carboxylate, sodium PEG-7 olive oil carboxylate,
phosphoric esters and salts, such as, for example, DEA oleth-10 phosphate and dilaureth4 phosphate,
alkylsulfonates, for examples sodium cocomonoglyceride sulfate, sodium C₁₂₋₁₄ olefinsulfonate, sodium lauryl sulfoacetate and magnesium PEG-3 cocamide sulfate.

Further advantageous amphoteric surfactants are
N-alkylamino acids, for example aminopropylalkylglutamide, alkylaminopropionic acid, sodium alkylimidodipropionate and lauroamphocarboxyglycinate.

Further advantageous nonionic surfactants are alcohols.

Further suitable anionic surfactants for the purposes of the present invention are also
acyl glutamates, such as di-TEA palmitoyl aspartate and sodium caprylic/capric glutamate,
acylpeptides, for example palmitoyl hydrolyzed milk protein, sodium cocoyl hydrolyzed soya protein and sodium/potassium cocoyl hydrolyzed collagen,
   and carboxylic acids and derivatives, such as
for example lauric acid, aluminun stearate, magnesium alkanolate and zinc undecylenate,
ester carboxylic acids, for example calcium stearoyl lactylate, laureth-6 citrate and sodium PEG4 lauramide carboxylate,
alkylarylsulfonates.

Further suitable cationic surfactants for the purposes of the present invention are also
alkylamines,
alkylimidazoles, and
ethoxylated amines.

Further suitable nonionic surfactants for the purposes of the present invention are also amine oxides, such as cocoamidopropylamine oxide.

It is advantageous for the purposes of the present invention if the content of one or more washing-active surfactants in the cosmetic preparation is chosen from the range from 0.1 to 25% by weight, very particularly advantageously from 10 to 20% by weight, in each case based on the total weight of the preparation.

In addition, according to the invention, polysorbates can advantageously be incorporated as washing-active agents.

Besides one or more water phases, the cosmetic preparation according to the invention can additionally comprise one or more oil phases and be present, for example, in the form of W/O, O/W, W/O/W or O/W/O emulsions. Such emulsions may preferably also be a microemulsion (e.g. a PIT emulsion) or a solids emulsions (i.e. an emulsion which is stabilized by solids, e.g. a Pickering emulsion). The type of emulsion can be established as desired using the emulsifiers and emulsifier mixtures known sufficiently to the person skilled in the art.

Besides water, the preparation according to the invention can also comprise other ingredients as aqueous phase of the emulsion in accordance with the invention, for example alcohols, diols, or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethyleneglycol monomethyl or monoethyl ether and analogous products, also alcohols of low carbon number, e.g. ethanol, isopropanol, 1,2-propanediol and glycerol.

The oil phase of the cosmetic or dermatological cleansing emulsions for the purposes of the present invention is advantageously chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, of silicone oils, of dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24, in particular 12 to 18, carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

Advantageously, the oil phase can also have a content of cyclic or linear silicone oils or consist entirely of such oils, although it is preferred to use an additional content of other oil phase components apart from the silicone oil or the silicone oils.

Cyclomethicone (octamethylcyclotetrasiloxane) is advantageously used as silicone oil to be used in accordance with the invention. However, other silicone oils are also to be used advantageously for the purposes of the present invention, for example hexamethylcyclotrisiloxane, polydimethylsiloxane, poly(methylphenylsiloxane).

In addition, mixtures of cyclomethicone and isotridecylisononanoate, cyclomethicone and 2-ethylhexyl isostearate are particularly advantageous.

The oil phase is also advantageously chosen from the group of phospholipids. The phospholipids are phosphoric esters of acylated glycerols. Of greatest importance among the phosphatidylcholines are, for example, the lecithins, which are characterized by the general structure 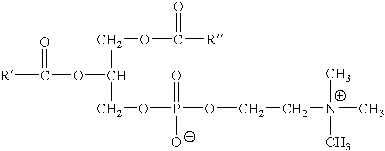
where R′ and R″ are typically unbranched aliphatic radicals having 15 or 17 carbon atoms and up to 4 cis double bonds.

It is advantageous according to the invention if the cosmetic cleansing emulsion comprises one or more polyacrylates.

Advantageous polyacrylates according to the invention are polymers of acrylic acid, in particular those which are chosen from the group of so-called carbomers or carbopols (Carbopol® is actually a registered trade mark of B.F. Goodrich Company). Polyacrylates are compounds of the general structural formula 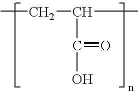
whose molecular weight can be between about 400,000 and more than 4,000,000. The group of polyacrylates also includes acrylate-alkyl acrylate copolymers, for example those which are characterized by the following structure: 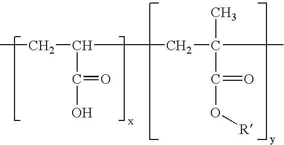
Here, R′ is a long-chain alkyl radical, and x and y are numbers which symbolize the particular stoichiometric fraction of each of the comonomers. These polyacrylates are also advantageous for the purposes of the present invention.

Advantageous carbopols are, for example, the grades 907, 910, 934, 940, 941, 951, 954, 980, 981, 1342, 1382, 2984 and 5984 and also the ETD (easy-to-disperse) grades 2001, 2020, 2050, where these compounds may be present individually or in any combinations with one another.

Also advantageous for the purposes of the present invention are the copolymers of C₁₀₋₃₀-alkyl acrylates and one or more monomers of acrylic acid, of methacrylic acid or esters thereof comparable with the acrylate-alkyl acrylate copolymers. The INCI name for such compounds is “Acrylates/C 10-30 Alkyl Acrylate Crosspolymer”. Those available under the tradenames Pemulen TR1 and Pemulen TR2 from B.F. Goodrich Company are particularly advantageous.

It is particularly advantageous according to the invention if the polyacrylates used are C₁₀ to C₃₀-alkyl acrylate copolymers.

It is advantageous for the purposes of the present invention if the content of one or more polyacrylates in the cosmetic or dermatological cleansing emulsion is chosen from the range from 0.5 to 2% by weight, very particularly advantageously from 0.7 to 1.5% by weight, in each case based on the total weight of the preparation.

Apart from the abovementioned substances, the compositions according to the invention optionally comprise the additives customary in cosmetics, for example perfume, dyes, antimicrobial substances, emulsifiers, re-fatting agents, complexing and sequestering agents, pearlizing agents, plant extracts, vitamins, active ingredients, preservatives, bactericides, pigments which have a coloring effect, thickeners, antiperspirants, bleaches, self-tanning agents, repellents, film formers, softeners, moisturizers and/or humectants, or other customary constituents of a cosmetic or dermatological formulation, such as alcohols, polyols, polymers, foam stabilizers, electrolytes, organic solvents or silicone derivatives.

An additional content of antioxidants is generally preferred. According to the invention, favorable antioxidants which may be used are all antioxidants which are customary or suitable for cosmetic and/or dermatological application.

The amount of antioxidants (one or more compounds) in the preparations is preferably 0.001 to 10% by weight, particularly preferably 0.05-7% by weight, in particular 0.1-5% by weight, based on the total weight of the preparation.

If vitamin E and/or derivatives thereof is/are the antioxidant(s), it is advantageous to choose their particular concentrations from the range 0.001-5% by weight, based on the total weight of the formulation.

If vitamin A or vitamin A derivatives, or carotenes or derivatives thereof is/are the antioxidant(s), it is advantageous to choose their particular concentrations from the range 0.001-5% by weight, based on the total weight of the formulation.

Moreover, selected inventive formulations which comprise, for example, known antiwrinkle active ingredients, such as flavone glycosides (in particular α-glycosylrutin), coenzyme Q10, vitamin E and/or derivatives and the like, are particularly advantageously suitable for the prophylaxis and treatment of cosmetic or dermatological changes in the skin, as arise, for example, during skin aging (e.g. lines and wrinkles). Furthermore, they are advantageously suitable to counter the appearance of dry and/or rough skin.

If α-glycosylrutin is the antioxidant, it is advantageous to choose its particular concentrations from the range 0.001-5% by weight, based on the total weight of the formulation.

A content of UV light protection filters is also advantageous for the purposes of the present invention, it being possible to incorporate all organic and inorganic (micropigments) UV light protection filters approved according to the positive list of the Cosmetics Directive (e.g. Annex 7 of the Cosmetics Directive) in any mixtures and amounts into the preparation. Suitable inorganic micropigments are, for example, the oxides of titanium, zinc, iron, zirconium, silicon, manganese, aluminum and cerium, and the sulfate of barium. These micropigments may also be surface-coated by conventional methods.

Advantageous organic UV light protection filters according to the invention are, for example, the derivatives of cinnamic acid, salicylic acid, aminobenzoic acid, of benzoazolyls, benzodiazolyls, benzotriazoles, benzophenones, benzimidazoles (here in particular the sulfonic acid derivatives and salts), triazines, and trisiloxanes, phenyl acrylates, and camphor derivatives.

The active ingredients, auxiliaries and additives which can be used advantageously according to the invention here are in no way limited to the substances and compounds mentioned here by name.

The invention also provides the process for the preparation of a water-insoluble substrate impregnated with a cosmetic preparation, which is characterized in that the cosmetic preparation is sprayed onto the substrate or the substrate is immersed into a bath containing the cosmetic preparation.

The invention further provides the process for the preparation of a water-insoluble substrate impregnated with a cosmetic preparation, characterized in that the substrate is dried following impregnation with the cosmetic preparation.

Last but not least, the invention provides the use of the water-insoluble substrate impregnated with a cosmetic preparation as a cleansing or care wipe which appears dry or wet. In particular, the water-insoluble substrate according to the invention is suitable for face cleansing and babycare.

The following examples, which describe the composition of the cosmetic preparations according to the invention, are intended to illustrate the invention, but are not intended to limit it to these examples. The numerical values in the examples are percentages by weight, based on the total weight of the particular preparation.

### EXAMPLES 1-27

Formulations for Water-Insoluble Substrates Which Appear Dry

| | 1 | 2 | 3 | 4 | 5 |
|---|---|---|---|---|---|
| Decyl glucoside | 20.0% | 25.0% | 7.0% | 20.0% | 10.0% |
| Sodium cocoyl glutamate | — | — | — | 5.0% | 10.0% |
| Carbopol 1382 | 2.0% | 2.5% | 3.0% | — | 1.2% |
| Hydroxypropyl starch | 2.0% | 1.0% | 10.0% | 4.0% | 7.0% |
| phosphate ester | | | | | |
| (Structure XL) | | | | | |
| Sodium hydroxide | 0.5% | 1.0% | 0.5% | 1.0% | 0.5% |
| Butylene glycol | 15.0% | 10.0% | 20.0% | 10.0% | — |
| Propylene glycol | 30.0% | 17.5% | 20.0% | 20.0% | — |
| Na₃HEDTA | 0.5% | 0.5% | 0.5% | 1.0% | 0.5% |
| Sodium benzoate | 0.3% | 0.3% | 0.3% | 0.3% | 0.3% |
| Sodium salicylate | 0.2% | 0.4% | 0.2% | 0.2% | 0.2% |
| Perfume | q.s. | q.s. | q.s. | q.s. | q.s. |
| Water | ad 100 | ad 100 | ad 100 | ad 100 | ad 100 |
| | 6 | 7 | 8 | 9 | 10 |
| Sodium laureth sulfate | 4.0% | 5.0% | 7.0% | 7.0% | — |
| Methyl cocoyl taurate | 0.6% | 1.5% | 0.6% | 3.0% | 6.0% |
| Hydroxypropyl starch | 2.0% | 3.0% | 10.0% | 5.0% | 8.0% |
| phosphate ester | | | | | |
| (Structure XL) | | | | | |
| Carbopol 980 | 1.2% | 0% | 1.2% | 0.5% | 0.6% |
| Sodium hydroxide | 0.5% | 0.5% | 1.0% | 0.5% | 0.5% |
| Glycerol | 2.0% | 5.0% | 10.0% | — | 2.0% |
| Xanthan gum | 0.3% | 0.1% | 2.0% | — | — |
| Phenoxyethanol | 0.5% | 0.5% | 0.5% | 0.5% | 0.5% |
| Parabens | 0.2% | 0.2% | 0.2% | 0.2% | 0.2% |
| Perfume | q.s. | q.s. | q.s. | q.s. | q.s. |
| Water | ad 100 | ad 100 | ad 100 | ad 100 | ad 100 |
| | 11 | 12 | 13 | 14 | 15 |
| Sodium laureth sulfate | 14.0% | 11.0% | 20.0% | 5.0% | — |
| Cocoamidopropylbetaine | 1.7% | 7.0% | 7.5% | — | — |
| Sodium cocoyl glutamate | 1.4% | 0.75% | 5.0% | 2.0% | 10.0% |
| Hydroxypropyl starch | 4.0% | 15.0% | 8.0% | 3.0% | 5.0% |
| phosphate ester | | | | | |
| (Structure XL) | | | | | |
| PEG-40 hydrogenated | 0.5% | 0.5% | 1.0% | 0.2% | 0.5% |
| castor oil | | | | | |
| PEG-100 hydrogenated | 0.5% | 1.0% | 0.5% | — | 0.5% |
| glyceryl palmitate | | | | | |
| Polyquaternium-10 | 0.2% | — | 0.2% | — | 0.5% |
| Sodium benzoate | 0.5% | 0.5% | 0.5% | 0.5% | 0.5% |
| Sodium salicylate | 0.2% | 0.2% | 0.2% | 0.2% | 0.2% |
| Citric acid | 0.5% | 0.5% | 0.5% | 0.5% | 0.5% |
| Perfume | q.s. | q.s. | q.s. | q.s. | q.s. |
| Water | ad 100 | ad 100 | ad 100 | ad 100 | ad 100 |
| | 16 | 17 | 18 | 19 | 20 |
| Sodium myreth sulfate | 10.0% | 8.0% | 13.0% | — | — |
| Sodium cocoyl glutamate | 5.0% | 2.0% | 7.0% | 3.0% | 2.2% |
| Decyl glucoside | — | 3.0% | — | 3.0% | 2.0% |
| Sodium | 6.5% | 10% | 8.0% | — | — |
| cocoamphoacetate | | | | | |
| Hydroxypropyl starch | 2.0% | 0.8% | 5.0% | 2.0% | 0.3% |
| phosphate ester | | | | | |
| (Structure XL) | | | | | |
| PEG-200 hydrogenated | 0.4% | 1.0% | 0.4% | — | — |
| glyceryl palmitate | | | | | |
| PEG-40 hydrogenated | 1.0% | 1.0% | 1.0% | 0.5% | 0.5% |
| castor oil | | | | | |
| Diammonium citrate | 0.2% | 0.1% | 0.2% | 0.1% | 0.1% |
| Polyquaternium-10 | 0.2% | — | — | — | 0.5% |
| Sodium benzoate | 0.3% | 0.3% | 0.3% | 0.3% | 0.3% |
| Sodium salicylate | 0.2% | 0.2% | 0.2% | 0.2% | 0.2% |
| Citric acid | 1.2% | 1.2% | 1.2% | 0.8% | 1.0% |
| Perfume | q.s. | q.s. | q.s. | q.s. | q.s. |
| Water | ad 100 | ad 100 | ad 100 | ad 100 | ad 100 |

| | 21 | 22 | 23 | 24 |
|---|---|---|---|---|
| Sodium cocoyl glutamate | 5.0% | — | — | — |
| Sodium lauryl ether sulfate | — | 7.0% | — | — |
| Sodium lauroyl sarcosinate | — | — | 10.0% | — |
| Sodium myristyl ether sulfate | — | — | — | 9.0% |
| Decyl glucoside | 6.0% | 8% | — | — |
| Lauryl glucoside | — | — | 6.0% | 6.0% |
| Hydroxypropyl starch phosphate | 0.4% | 0.4% | 0.8% | 0.2% |
| ester (Structure XL) | | | | |
| Polyquaternium-10 | 0.2% | — | — | 0.1% |
| Guar hydroxypropyltrimonium | — | 0.2% | — | — |
| chloride | | | | |
| Polyquaternium-22 | — | — | 0.2% | — |
| PEG-200 hydrogenated glyceryl | 0.5% | — | — | — |
| palmitate | | | | |
| PEG-40 hydrogenated castor oil | 0.1% | 0.2% | 0.1% | 0.1% |
| PEG-100 hydrogenated glyceryl | — | 0.5% | — | 0.5 |
| palmitate | | | | |
| Sodium benzoate | 0.5% | 0.5% | — | 0.5% |
| Sodium salicylate | — | 0.2% | — | 0.2% |
| Methyldibromoglutaronitrile | — | — | 0.1% | — |
| Phenoxyethanol | — | — | 0.2% | — |
| Jojoba oil (Buxus chinensis) | 0.5% | — | — | — |
| Citric acid | q.s. | q.s. | 0.2% | q.s. |
| Perfume | q.s. | q.s. | q.s. | q.s. |
| Water | ad 100 | ad 100 | ad 100 | ad 100 |

### EXAMPLE 25

Emulsion

| | Amount | Amount |
|---|---|---|
| Constituent | % by wt. | % by wt. |
| Hydroxypropyl starch phosphate | 5.0 | 10.0 |
| ester (Structure XL) | | |
| Paraffinum liquidum | 1.0 | 8.0 |
| Glycerol | 14.0 | 5.0 |
| Octyl stearate | 2.0 | 2.0 |
| Glyceryl stearate, ceteareth-20, | 6.0 | 1.5 |
| ceteareth-12, cetearyl alcohol, | | |
| cetyl palmitate | | |
| Phenoxyethanol, | 0.5 | 0.5 |
| methylparaben, ethylparaben, | | |
| propylparaben, butylparaben, | | |
| isobutylparaben | | |
| Perfume | q.s. | q.s. |
| Ceteareth-20 | 20.0 | 0.3 |
| Methylparaben | 1.0 | 0.3 |
| Water | ad 100.0 | ad 100.0 |

### EXAMPLE 26

Aqueous Impregnation Solution

| | Amount | Amount |
|---|---|---|
| Constituent | % by wt. | % by wt. |
| Hydroxypropyl starch phosphate | 10.0 | 20.0 |
| ester (Structure XL) | | |
| Butylene glycol | 1.0 | 1.0 |
| PEG-40 hydrogenated castor oil | 1.0 | 0.8 |
| Phenoxyethanol, | 1.5 | 0.7 |
| methylparaben, ethylparaben, | | |
| propylparaben, butylparaben, | | |
| isobutylparaben | | |
| Potassium sorbate | 0.5 | 0.3 |
| Perfume | q.s. | q.s. |
| Citric acid | 0.5 | 0.2 |
| Water | ad 100.0 | ad 100.0 |

### EXAMPLE 27

Alcoholic Impregnation Solution

| | Amount | Amount |
|---|---|---|
| Constituent | % by wt. | % by wt. |
| Hydroxypropyl starch phosphate | 8.0 | 15.0 |
| ester (Structure XL) | | |
| Ethanol | 60.0 | 60.0 |
| Glycerol | 5.0 | 15.0 |
| Isopropyl alcohol | — | 5.0 |
| Ethylenediamine | — | 1.0 |
| Dexpanthenol | — | 1.0 |
| Carbomer | — | 3.0 |
| Perfume | q.s. | q.s. |
| Dye | 0 | 0.5 |
| Water | ad 100.0 | ad 100.0 |

### EXAMPLES 28-39

Formulations for Water-insoluble Substrates Which Appear Wet

### EXAMPLE 28

| | Amount |
|---|---|
| Constituent | % by wt. |
| Hydroxypropyl starch phosphate | 0.5 |
| ester (Structure XL) | |
| Perfume | q.s. |
| Paraffinum liquidum | ad 100.0 |

### EXAMPLE 29

Emulsion

| | Amount | Amount |
|---|---|---|
| Constituent | % by wt. | % by wt. |
| Hydroxypropyl starch phosphate | 8.0 | 3.0 |
| ester (Structure XL) | | |
| Paraffinum liquidum | 0.5 | 8.0 |
| Glycerol | 7.0 | 5.0 |
| Octyl stearate | 1.0 | 2.0 |
| Glyceryl stearate, ceteareth-20, | 3.0 | 1.5 |
| ceteareth-12, cetearyl alcohol, | | |
| cetyl palmitate | | |
| Phenoxyethanol, | 0.5 | 0.5 |
| methylparaben, ethylparaben, | | |
| propylparaben, butylparaben, | | |
| isobutylparaben | | |
| Perfume | q.s. | q.s. |
| Ceteareth-20 | 10.0 | 0.3 |
| Methylparaben | 1.0 | 0.3 |
| Water | ad 100.0 | ad 100.0 |

### EXAMPLE 30

Aqueous Impregnation Solution

| | Amount | Amount |
|---|---|---|
| Constituent | % by wt. | % by wt. |
| Hydroxypropyl starch phosphate | 10.0 | 7.0 |
| ester (Structure XL) | | |
| Butylene glycol | 1.0 | 1.0 |
| PEG-40 hydrogenated castor oil | 1.0 | 0.8 |
| Phenoxyethanol, | 1.5 | 0.7 |
| methylparaben, ethylparaben, | | |
| propylparaben, butylparaben, | | |
| isobutylparaben | | |
| Potassium sorbate | 0.5 | 0.3 |
| Perfume | q.s. | q.s. |
| Citric acid | 0.5 | 0.16 |
| Water | ad 100.0 | ad 100.0 |

### EXAMPLE 31

| | Amount |
|---|---|
| Constituent | % by wt. |
| Hydroxypropyl starch | 0.5 |
| phosphate ester (Structure | |
| XL) | |
| Dimethicone | 20.0 |
| Silicone gum | 7.0 |
| Phenyltrimethylmethicone | 7.0 |
| Perfume | q.s. |
| Cyclomethicone | ad 100.0 |

### EXAMPLE 32

Alcoholic Impregnation Solution

| | Amount | Amount |
|---|---|---|
| Constituent | % by wt. | % by wt. |
| Hydroxypropyl starch phosphate | 2.0 | 8.0 |
| ester (Structure XL) | | |
| Ethanol | 60.0 | 60.0 |
| Glycerol | 5.0 | 5.0 |
| Isopropyl alcohol | 0 | 5.0 |
| Ethylenediamine | 0 | 1.0 |
| Dexpanthenol | 0 | 1.0 |
| Carbomer | 0 | 3.0 |
| Perfume | q.s. | q.s. |
| Dye | 0 | 0.5 |
| Water | ad 100.0 | ad 100.0 |

### EXAMPLE 33

Aftersun/Suncare Emulsion

| | Amount |
|---|---|
| Constituent | % by wt. |
| Hydroxypropyl starch | 5.0 |
| phosphate ester (Structure | |
| XL) | |
| Ceteth-15 | 6.0 |
| Glyceryl isostearate | 2.0 |
| Cetyl alcohol | 1.0 |
| Dicaprylyl carbonate | 5.0 |
| Octyldodecanol | 3.0 |
| Cyclomethicone | 1.0 |
| Butylene glycol | 3.0 |
| Ethanol | 5.0 |
| DMDM hydantoin | 0.6 |
| Octoxyglycerol | 1.0 |
| Antioxidants | 0.5 |
| Perfume | q.s. |
| Dye | 0.3 |
| Water | ad 100 |

### EXAMPLE 34

Nongreasy Bodycare Emulsion

| | Amount |
|---|---|
| Constituent | % by wt. |
| Hydroxypropyl starch | 4.0 |
| phosphate ester (Structure XL) | |
| Ceteareth-12 | 6.0 |
| Glyceryl stearate | 3.5 |
| Cetyl palmitate | 3.0 |
| Dicaprylyl ether | 5.0 |
| Cyclomethicone | 3.0 |
| Phenyltrimethicone | 1.0 |
| Paraffin wax | 2.0 |
| Glycerol | 7.5 |
| Parabens | 1.0 |
| Phenoxyethanol | 1.0 |
| AGR | 0.5 |
| Perfume | q.s. |
| Dyes | 0.5 |
| Water | ad 100 |

### EXAMPLE 35

Sunscreen Composition for Silky Skin Feel

| | Amount | Amount |
|---|---|---|
| Constituent | % by wt. | % by wt. |
| Hydroxypropyl starch phosphate | 3.6 | 6.0 |
| ester (Structure XL) | | |
| Ceteareth-20 | 5.5 | 6.5 |
| Glyceryl stearate | 4.0 | 2.0 |
| Stearyl alcohol | 3.0 | 1.0 |
| Dicaprylyl ether | 5.0 | 5.0 |
| Octyldodecanol | 3.0 | 3.0 |
| Phenyltrimethicone | 1.0 | — |
| Bisethylhexyloxyphenol | 2.0 | — |
| methoxyphenyltriazine | | |
| Octocrylene | 7.0 | 7.0 |
| Diethylhexylbutamidotriazone | 1.0 | — |
| Ethylhexyl methoxycinnamate | 4.0 | 4.0 |
| Butylene glycol | 1.0 | — |
| Vitamin E acetate | 1.0 | — |
| C12-15 alkyl benzoates | — | 1.0 |
| Titanium dioxide | — | 2.0 |
| Bisethylhexyloxyphenol | — | 2.0 |
| methoxyphenyltriazine | | |
| PVP/hexadecene copolymer | 1.0 | — |
| Parabens | 1.0 | 1.0 |
| Antioxidants | 0.5 | 0.5 |
| Perfume | q.s. | q.s. |
| Water | ad 100 | ad 100 |

### EXAMPLE 36

Sunscreen Formulation

| | Amount |
|---|---|
| Constituent | % by wt. |
| Hydroxypropyl starch phosphate | 5.0 |
| ester (Structure XL) | |
| Steareth-20 | 6.5 |
| Glyceryl isostearate | 2.0 |
| Cetyl alcohol | 1.0 |
| Dicaprylyl carbonate | 5.0 |
| Shea butter | 3.0 |
| C12-15 alkyl benzoates | 1.0 |
| Bisethylhexyloxyphenol | 2.0 |
| methoxyphenyltriazine | |
| Butylmethoxydibenzoylmethane | 1.0 |
| Ethylhexyltriazone | 2.0 |
| Phenylbenzimidazolesulfonic | 2.0 |
| acid | |
| Ethylhexyl methoxycinnamate | 4.0 |
| Glycerol | 10.0 |
| Tricontanyl PVP | 1.0 |
| Citrate buffer | 1.0 |
| Parabens | 1.0 |
| Antioxidants | 0.5 |
| Perfume | q.s. |
| Water | ad 100 |

### EXAMPLE 37

Sunscreen Formulation

| | Amount |
|---|---|
| Constituent | % by wt. |
| Hydroxypropyl starch phosphate | 3.0 |
| ester (Structure XL) | |
| Ceteareth-30 | 7.0 |
| Glyceryl isostearate | 2.5 |
| Cetyl alcohol | 1.0 |
| Dicaprylyl carbonate | 4.0 |
| Capric/caprylic triglyceride | 2.0 |
| C12-15 alkyl benzoates | 6.0 |
| Methylene bisbenzotriazolyl | 2.0 |
| tetramethylbutylphenol | |
| Butylmethoxydibenzoylmethane | 2.0 |
| Ethylhexyl triazone | 4.0 |
| Bisimidazylate | 2.0 |
| Methylbenzylidenecamphor | 4.0 |
| Glycerol | 5.0 |
| PVP hexadecene copolymer | 1.0 |
| Parabens | 1.0 |
| Antioxidants | 0.5 |
| Perfume | q.s. |
| Water | ad 100 |

### EXAMPLE 38

Sunscreen Formulation

| | Amount |
|---|---|
| Constituent | % by wt. |
| Hydroxypropyl starch phosphate | 4.0 |
| ester (Structure XL) | |
| Ceteareth-20 | 7.5 |
| Glyceryl stearate | 3.0 |
| Cetyl palmitate | 1.5 |
| Dicaprylyl carbonate | 5.0 |
| Cocoglycerides | 2.0 |
| C12-15 alkyl benzoates | 6.0 |
| Barium sulfate | 2.0 |
| Bisethylhexyloxyphenol | 2.0 |
| methoxyphenyltriazine | |
| Ethylhexyltriazone | 4.0 |
| Bisimidazylate | 1.0 |
| Phenylbenzimidazolesulfonic acid | 2.0 |
| Methylbenzylidenecamphor | 4.0 |
| PVP hexadecene copolymer | 1.0 |
| NaOH | 0.5 |
| Parabens | 1.0 |
| Antioxidants | 0.5 |
| Perfume | q.s. |
| Water | ad 100 |

### EXAMPLE 39

Aftersun/Skincare Formulation

| | Amount |
|---|---|
| Constituent | % by wt. |
| Hydroxypropyl starch phosphate | 10.0 |
| ester (Structure XL) | |
| Ceteth-15 | 6.0 |
| Glyceryl isostearate | 2.0 |
| Cetyl alcohol | 1.0 |
| Dicaprylyl carbonate | 5.0 |
| Shea Butter | 1.0 |
| Octyldodecanol | 3.0 |
| Cylomethicone | 1.0 |
| Mineral Oil | 2.0 |
| Ethanol | 5.0 |
| Parabens | 1.0 |
| Antioxidants | 0.5 |
| Perfume | q.s. |
| Water | ad 100 |

### EXAMPLE 40

Oil Formulation

| | Amount |
|---|---|
| Constituent | % by wt. |
| Hydroxypropyl starch phosphate | 0.5 |
| ester (Structure XL) | |
| Capric/caprylic triglyceride | 2.0 |
| C12-15 alkyl benzoates | 6.0 |
| Butylmethoxydibenzoylmethane | 2.0 |
| Ethylhexyltriazone | 2.0 |
| Bisethylhexyloxyphenol | 1.0 |
| methoxyphenyltriazine | |
| Methylbenzylidenecamphor | 4.0 |
| Shea butter | 1.0 |
| Butylene glycol | 3.0 |
| dicaprate/dicaprylate | |
| Dimethicone | 5.0 |
| Parabens | 1.0 |
| Antioxidants | 0.5 |
| Perfume | q.s. |
| Mineral oil | ad 100 |

### EXAMPLE 41

Oil Formulation

| | Amount/% | Amount/% | Amount/% |
|---|---|---|---|
| Constituent | by wt. | by wt. | by wt. |
| Hydroxypropyl starch phosphate | 0.5 | 0.5 | 1.0 |
| ester (Structure XL) | | | |
| Dicaprylyl carbonate | 5.0 | 5.0 | 5.0 |
| Bisethylhexyloxyphenol | 2.0 | 2.0 | — |
| methoxyphenyltriazine | | | |
| Ethylhexyltriazone | 4.0 | — | — |
| Methylbenzylidenecamphor | 4.0 | — | — |
| Shea butter | 1.0 | 1.0 | 1.0 |
| Octyldodecanol | 3.0 | — | — |
| Cylomethicone | 1.0 | — | — |
| Diethylhexylbutamidotriazone | — | 4.0 | — |
| Methylbenzylidenecamphor | — | 1.0 | — |
| Ethylhexyl methoxycinnamate | — | — | 10.0 |
| Bisethylhexyloxyphenol | — | — | 2.0 |
| methoxyphenyltriazine | | | |
| Diethylhexylbutamidotriazone | — | — | 4.0 |
| Octocrylene | — | — | 5.0 |
| Phenyltrimethicone | — | 1.0 | 1.0 |
| Vitamin E | 1.0 | 2.0 | 1.0 |
| Perfume | q.s. | q.s. | q.s. |
| Mineral oil | ad 100 | ad 100 | ad 100 |

### EXAMPLE 42

Aqueous Formulation

| | Amount |
|---|---|
| Constituent | % by wt. |
| Hydroxypropyl starch phosphate | 10.0 |
| ester (Structure XL) | |
| Bisimidazylate | 1.0 |
| Phenylbenzimidazolesulfonic acid | 2.0 |
| Glycerol | 10.0 |
| Parabens | 1.0 |
| Antioxidants | 0.5 |
| Perfume | q.s. |
| Water | ad 100 |

### EXAMPLES 43-66

Surfactant Formulations (Amount % By Wt.):

| | 43 | 44 | 45 | 46 | 47 |
|---|---|---|---|---|---|
| Decyl glucoside | 10.0% | 15.0% | 3.0% | 10.0% | 5.0% |
| Sodium cocoyl glutamate | — | — | — | 2.0% | 5.0% |
| Carbopol 1382 | 1.0% | 1.0% | 1.5% | 1.0% | 0.6% |
| Hydroxypropyl starch | 1.0% | 1.0% | 5.0% | 1.0% | 7.0% |
| phosphate ester | | | | | |
| (Structure XL) | | | | | |
| Sodium hydroxide | 0.5% | 0.5% | 0.5% | 0.5% | 0.5% |
| Butylene glycol | 10.0% | 10.0% | 10.0% | 10.0% | — |
| Propylene glycol | 17.5% | 17.5% | 17.5% | 17.5% | — |
| Na₃HEDTA | 0.5% | 0.5% | 0.5% | 0.5% | 0.5% |
| Sodium benzoate | 0.3% | 0.3% | 0.3% | 0.3% | 0.3% |
| Sodium salicylate | 0.2% | 0.2% | 0.2% | 0.2% | 0.2% |
| Perfume | q.s. | q.s. | q.s. | q.s. | q.s. |
| Water | ad 100 | ad 100 | ad 100 | ad 100 | ad 100 |
| | 48 | 49 | 50 | 51 | 52 |
| Sodium laureth sulfate | 2.0% | 2.0% | 7.0% | 7.0% | — |
| Methyl cocoyl taurate | 0.6% | 0.6% | 0.6% | 0.6% | 6.0% |
| Hydroxypropyl starch | 1.0% | 3.0% | 1.0% | 5.0% | 8.0% |
| phosphate ester | | | | | |
| (Structure XL) | | | | | |
| Carbopol 980 | 1.2% | 1.2% | 1.2% | 0.5% | 0.6% |
| Sodium hydroxide | 0.5% | 0.5% | 0.5% | 0.5% | 0.5% |
| Glycerol | 2.0% | 2.0% | 2.0% | — | 2.0% |
| Xanthan gum | 0.25% | 0.1% | 0.3% | — | — |
| Phenoxyethanol | 0.5% | 0.5% | 0.5% | 0.5% | 0.5% |
| Parabens | 0.2% | 0.2% | 0.2% | 0.2% | 0.2% |
| Perfume | q.s. | q.s. | q.s. | q.s. | q.s. |
| Water | ad 100 | ad 100 | ad 100 | ad 100 | ad 100 |
| | 53 | 54 | 55 | 56 | 57 |
| Sodium laureth sulfate | 13.2% | 11.0% | 9.5% | 5.0% | — |
| Cocoamidopropylbetaine | 1.7% | 3.3% | 3.8% | — | — |
| Sodium cocoyl glutamate | 1.3% | 0.8% | 2.5% | 2.0% | 7.0% |
| Hydroxypropyl starch | 2.0% | 8.0% | 4.0% | 3.0% | 2.0% |
| phosphate ester | | | | | |
| (Structure XL) | | | | | |
| PEG-40 hydrogenated | 0.5% | 0.5% | 0.5% | 0.1% | 0.5% |
| castor oil | | | | | |
| PEG-100 hydrogenated | 0.5% | 0.5% | 0.5% | — | 0.5% |
| glyceryl palmitate | | | | | |
| Polyquaternium-10 | 0.2% | — | 0.2% | — | 0.2% |
| Sodium benzoate | 0.4% | 0.4% | 0.5% | 0.5% | 0.4% |
| Sodium salicylate | 0.2% | 0.2% | 0.2% | 0.2% | 0.2% |
| Citric acid | 0.5% | 0.5% | 0.5% | 0.5% | 0.5% |
| Perfume | q.s. | q.s. | q.s. | q.s. | q.s. |
| Water | ad 100 | ad 100 | ad 100 | ad 100 | ad 100 |
| | 58 | 59 | 60 | 61 | 62 |
| Sodium myreth sulfate | 5.0% | 4.0% | 6.0% | — | — |
| Sodium cocoyl glutamate | 2.5% | 1.0% | 5.0% | 3.0% | 1.8% |
| Decyl glucoside | — | 3.0% | — | 3.0% | 2.0% |
| Sodium | 6.5% | 7.0% | 8.0% | — | — |
| cocoamphoacetate | | | | | |
| Hydroxypropyl starch | 2% | 0.8% | 5.0% | 2.0% | 0.3% |
| phosphate ester | | | | | |
| (Structure XL) | | | | | |
| PEG-200 hydrogenated | 0.4% | 0.4% | 0.4% | — | — |
| glyceryl palmitate | | | | | |
| PEG-40 hydrogenated | 1.0% | 1.0% | 1.0% | 0.5% | 0.5% |
| castor oil | | | | | |
| Diammonium citrate | 0.2% | 0.1% | 0.1% | 0.2% | 0.2% |
| Polyquaternium-10 | 0.2% | — | — | — | 0.2% |
| Sodium benzoate | 0.3% | 0.3% | 0.3% | 0.3% | 0.3% |
| Sodium salicylate | 0.2% | 0.2% | 0.2% | 0.2% | 0.2% |
| Citric acid | 1.2% | 1.2% | 1.2% | 0.8% | 1% |
| Perfume | q.s. | q.s. | q.s. | q.s. | q.s. |
| Water | ad 100 | ad 100 | ad 100 | ad 100 | ad 100 |

| | 63 | 64 | 65 | 66 |
|---|---|---|---|---|
| Sodium cocoyl glutamate | 2.5 | — | — | — |
| Sodium lauryl ether sulfate | — | 3.5 | — | — |
| Sodium lauroyl sarcosinate | — | — | 5.0 | — |
| Sodium myristyl ether sulfate | — | — | — | 4.5 |
| Decyl glucoside | 3.0 | 4.0 | — | — |
| Lauryl glucoside | — | — | 3.0 | 3.0 |
| Hydroxypropyl starch phosphate ester (Structure | 0.2 | 0.4 | 0.8 | 0.2 |
| XL) | | | | |
| Polyquaternium-10 | 0.1 | — | — | 0.1 |
| Guar hydroxypropyltrimonium chloride | — | 0.15 | — | — |
| Polyquaternium-22 | — | — | 0.2 | — |
| PEG-200 hydrogenated glyceryl palmitate | 0.5 | — | — | — |

1. A cosmetic preparation-containing composition comprising:
a water-insoluble substrate; and
a cosmetic preparation impregnated on said substrate,
wherein said cosmetic preparation comprises one or more pregelatinized, crosslinked starch derivatives.

2. The composition of claim 1, wherein the water-insoluble substrate includes a nonwoven material.

3. The composition of claim 2, wherein the nonwoven material comprises a water-jet-consolidated or water-jet-impressed nonwoven material.

4. The composition of claim 2, wherein the nonwoven material comprises a mixture of 60 to 80% viscose fibers and 40 to 20% polyethylene terephthalate fibers.

5. The composition of claim 1, wherein the water-insoluble substrate includes one or more materials selected from the group consisting of viscose, cotton, cellulose, jute, hemp, sisal, silk, wool, polypropylene, polyester, polyethylene terephthalate, aramid, nylon, polyvinyl derivatives, polyurethanes, polylactide, polyhydroxyalkanoate, cellulose esters, polyethylene, and mineral fibers.

6. The composition of claim 1, wherein the water-insoluble substrate comprises a wipe.

7. The composition of claim 1, wherein the one or more pregelatinized, crosslinked starch derivatives are present in the cosmetic preparation at a concentration of from 0.1 to 20% by weight, based on the total weight of the cosmetic preparation.

8. The composition of claim 1, wherein the one or more pregelatinized, crosslinked starch derivatives include one or more hydroxypropylated phosphate esters.

9. The composition of claim 8, wherein the one or more pregelatinized, crosslinked starch derivatives include hydroxypropyl distarch phosphate.

10. The composition of claim 1, wherein the cosmetic preparation further comprises one or more washing-active surfactants.

11. The composition of claim 10, wherein the one or more washing-active surfactants are present in the cosmetic preparation at a concentration of from 0.1 to 25% by weight, based on the total weight of the cosmetic preparation.

12. The composition of claim 1, wherein the cosmetic preparation further comprises one or more oil phase components.

13. The composition of claim 1, wherein the cosmetic preparation further comprises one or more polyacrylates.

14. The composition of claim 1, wherein the cosmetic preparation further comprises one or more components selected from the group consisting of cosmetic or dermatological active ingredients, auxiliaries, and additives.

15. The composition of claim 1, wherein said composition is a cleansing or care wipe.

16. The composition of claim 15, wherein said cleansing or care wipe appears dry.

17. The composition of claim 16, wherein said cleansing or care wipe appears wet.

18. A process for the preparation of a cosmetic preparation-containing composition, comprising:
providing a water-insoluble substrate; and
impregnating the substrate with a cosmetic preparation comprising one or more pregelatinized, crosslinked starch derivatives.

19. The process of claim 18, wherein said impregnating step comprises spraying the cosmetic preparation onto the water-insoluble substrate.

20. The process of claim 18, wherein said impregnating step comprises immersing the water-insoluble substrate into a bath including the cosmetic preparation.

21. The process of claim 18, further comprising drying the water-insoluble substrate after said impregnation step.
